(19) United States
(12) Patent Application Publication
(10) Pub. No.: US 2015/0144488 A1
(43) Pub. Date: May 28, 2015

(54) METHOD AND APPARATUS FOR SPERM ENRICHMENT

(71) Applicant: FERTILITY INNOVATIONS LIMITED, Dorchester Dorset (GB)

(72) Inventor: Michael Howard James, Dorchester Dorset (GB)

(21) Appl. No.: 14/406,577

(22) PCT Filed: Jun. 13, 2013
(86) PCT No.: PCT/GB2013/051556

§ 371 Date: Dec. 9, 2014

(30) Foreign Application Priority Data
Jun. 13, 2012 (GB) 1210496.4

(51) Int. Cl. C12N 5/076

(52) U.S. Cl. CPC C12N 5/061
USPC 204/450; 204/600; 204/630

(57) ABSTRACT

Methods and apparatus for electrophoretic or electrophoretically assisted enrichment of sperm cells along a channel placed between two chambers. According to certain preferred embodiments, the methods and apparatuses may be used, simultaneous to sperm cell enrichment for the separation of said sperm cells from a pre-treatment chemical compound with which they have been contacted to improve their fertilization ability when subsequently used, for example, in intrauterine insemination or another method of insemination.

### PRIORITY STATEMENT

This application is a national stage application under 35 U.S.C. §371 of PCT International Application No. PCT/GB2013/051556, which has an international filing date of 13 Jun. 2013 and claims priority under 35 U.S.C. §119 to Great Britain Application No. 1210496.4 filed 13 Jun. 2012. The contents of each application recited above are incorporated herein by reference in their entirety.

### BACKGROUND

It is estimated that 1 in 7 human couples have problems conceiving. Estimates put the percentage of cases of infertility that can be ascribed to the male partner at between 25% and 50%, depending on the population. The principal cause of male infertility is poor semen quality. Poor semen quality can be caused by a reduced number of spermatozoa (“sperm cells”) and/or reduced motility (asthenozoospermia) of those cells that are present. A number of treatments are available for both male and female infertility including intracervical insemination (ICI) and intrauterine insemination (IUI). In essence, these procedures involve placing either recently collected sperm cells, pre-treated sperm cells or sperm cells which have been frozen and thawed into the cervix or uterus, typically after washing, by artificial means. ICI and IUI have great potential to be used in many cases of infertility currently treated by in vitro fertilisation (IVF) which is more invasive and more expensive. However, IUI and ICI typically suffer from low success rates primarily attributed to low semen quality.

Techniques for assisted conception are also important in veterinary medicine including livestock breeding and conservation biology, where poor animal fertility may be a barrier to commercial or conservation goals.

The chances of ICI or IUI treatment resulting in a pregnancy are greatly increased if the sperm cells used can be of the highest possible quality. Quality may be improved by any or all of the following:
pre “washing” of sperm, for example in Ham's F-10 media without L-glutamine, to remove seminal fluid, leukocytes and non-motile sperm;
separation techniques to enrich the sample with higher quality cells, for example by discontinuous gradient centrifugation or electrophoresis (see, for example, WO2005/033295 hereby incorporated by reference);
treatment of the cells ex-vivo with a chemical agent in order to increase motility or otherwise improve their fertilisation capacity.

Pre-treatment of cells with a chemical agent to improve fertilization capability is usually followed by a washing step both in order to prevent exposure of the female partner, zygote and embryo to the chemical agent and to prevent “burn out” of the sperm cells which may result if their motility is stimulated for too-prolonged a period of time such that when insemination takes place the sperm cells are “exhausted” or have undergone premature triggering of the acrosome reaction.

Common to all pre-treatment procedures is the imperative that the procedure be as fast as possible (because sperm quality decreases with time ex-vivo) and that it avoids as far as possible physical and chemical trauma (for example shearing forces) which may damage sperm cells.

It is also a key consideration of any ex-vivo manipulation of sperm cells that the number of separate manipulation steps should be minimised. This is because it is known that for each step in a manipulation a proportion of cells will be lost or have their viability compromised.

Dr. Josef Zech of Innsbruck has developed an apparatus for selecting motile sperm cells from non-motile sperm cells and other cell types. US 2005/0026274 and WO 2012/032165 (both of which are incorporated herein by reference) give further details of this device (herein referred to as the “Zech device”). In essence the Zech device consists of a first chamber into which a sample containing sperm cells (for example a sample of semen) is placed and a second chamber from which sperm cells may be removed for use, for example, in fertilisation. The two chambers are linked by a channel filled with medium such that motile sperm cells move along the channel from the first to the second chamber and less motile sperm cells are retained in the first chamber. US 2005/0026274 gives further details of the construction of the device and in particular of the channel which is dimensioned such that there is negligible movement of cells along it by convection processes.

The present invention is based on an adaptation of the Zech device and related methods and uses. By incorporating electrophoresis cell migration can be sped up, processing times reduced and the apparatus, device and uses simultaneously used for enriching sperm cells for motility, and separating sperm cells from a pre-treatment chemical compound to increase their fertilization capability.

### SUMMARY OF INVENTION

The invention provides in a first aspect, an apparatus for selecting sperm cells, comprising a first chamber for receiving the sperm cells to be selected, separate therefrom, a second chamber for receiving the selected sperm cells, and at least one channel extending from the first chamber to the second chamber and having an opening into the first chamber and into the second chamber at its respective ends, the channel being dimensioned such that when it is filled with a liquid medium there is negligible movement of sperm cells from the first chamber to the second chamber by convection currents in the liquid medium, but that motile sperm cells are able to swim through the liquid medium from the first chamber to the second chamber; characterised in that the apparatus is provided with a cathode in electrical communication with the first chamber and an anode in electrical communication with the second chamber such that a voltage may be provided between the first chamber and the second chamber thereby assisting the migration of motile sperm cells from the first chamber to the second chamber by electrophoresis.

The invention also provides a method of using an apparatus of the invention for separating motile sperm cells from non-motile sperm cells.

The invention also provides a method of treating sperm cells with a chemical compound in order to increase the sperm cells' fertilization capacity, comprising:
A) contacting the sperm cells with a chemical compound suitable for increasing the sperm cell's fertilization capacity in a first chamber of a sperm cell enrichment apparatus wherein said sperm cell enrichment apparatus comprises said first chamber for receiving sperm cells to be selected, separate therefrom a second chamber for receiving the selected sperm cells, and at least one channel extending from the first chamber to the second chamber and having an opening into the first chamber and into the second chamber at its respective ends, the or each channel being dimensioned such that when it is filled with a liquid medium there is negligible movement of sperm cells from the first chamber to the second chamber by convection currents in the liquid medium, but that motile sperm cells are able to swim through the liquid medium from the first chamber to the second chamber; and
B) applying a voltage between the first chamber and the second chamber such that the first chamber is placed at a more negative electrical potential than the second chamber;
C) waiting until at least a portion of motile sperm cells have migrated from the first chamber to the second chamber by swimming and/or electrophoresis wherein said chemical compound is retained in the first chamber or captured in a further chamber separate from the sperm cells in the second chamber.

The invention also provides use of a sperm cell separated from a chemical compound according to the method of the invention for use in intrauterine insemination (IIII), intra cervical insemination (ICI), in vitro fertilization (IVF) or intra cytoplasmic sperm injection (ICSI).

### BRIEF DESCRIPTION OF DRAWINGS

FIG. 1 is a diagrammatic representation of an apparatus of the invention.

FIG. 2 is a diagrammatic representation of an apparatus of the invention wherein the first chamber and the second chamber are connected by a bridge element.

FIG. 3 is a diagrammatic representation of an apparatus of the invention showing various alternative electrode configurations, and in the case of FIG. 3C, positioning of optional further chambers.

### DETAILED DESCRIPTION

The invention provides in a first aspect, an apparatus for selecting sperm cells, comprising a first chamber for receiving the sperm cells to be selected, separate therefrom, a second chamber for receiving the selected sperm cells, and at least one channel extending from the first chamber to the second chamber and having an opening into the first chamber and into the second chamber at its respective ends, the channel being dimensioned such that when it is filled with a liquid medium there is negligible movement of sperm cells from the first chamber to the second chamber by convection currents in the liquid medium, but that motile sperm cells are able to swim through the liquid medium from the first chamber to the second chamber; characterised in that the apparatus is provided with a cathode in electrical communication with the first chamber and an anode in electrical communication with the second chamber such that a voltage may be provided between the first chamber and the second chamber thereby assisting the migration of sperm cells from the first chamber to the second chamber by electrophoresis.

### Source of Sperm Cells

The various aspects of the invention primarily relate to humans. However, they may be applicable to other animals (especially other mammals) including livestock (especially horses, sheep, goats, cattle, pigs), racing animals (especially horses and camels), companion animals (including cats and dogs), wild animals (including big cats, antelopes and pandas) and research animals (including rodents such as rabbits, mice and rats).

### Apparatus for Simultaneous Separation of Chemical Compounds

Apparatus of the invention are suitable for selecting motile sperm cells from less motile sperm cells. However, they are also suitable for simultaneously separating sperm cells which have been pre-treated (either in the first chamber itself or prior to being placed in the first chamber) with a chemical compound from that chemical compound. In its simplest form, the apparatus needs no special modification. If the chemical compound has no electrical charge but is sufficiently large so as not to significantly migrate from the first chamber to the second chamber by convection or diffusion in a time-span sufficient for motile sperm cells to migrate from the first chamber to the second chamber by swimming and/or electrophoresis, then the apparatus need no further modification.

Further if the chemical compound is positively charged, the first chamber being at a more negative electrical potential will retain the chemical compound whilst the motile sperm cells migrate to the second chamber. Accordingly the apparatus of the invention may be especially suitable for separating sperm cells from positively charged chemical compounds.

If the cathode is not directly present in the first chamber but is located in a further chamber in electrical communication with the first chamber (for example via an ion-permeable membrane or a salt bridge), it is possible that the positively charged chemical compound will electrophoretically migrate into the further chamber. However, the principle remains the same in that the positively charged chemical compound is electrophoretically moved away from the channel(s) and the second chamber.

The invention also encompasses apparatus, methods and uses wherein the chemical compound is negatively charged. A negatively charged compound will electrophoretically migrate through the channel(s) into the second chamber along with motile sperm cells. Therefore, whilst the channel(s) is able to separate motile sperm cells from non-motile sperm cells, it is ineffective in separating motile sperm cells from the negatively charged chemical compound. This separation requires apparatus in accordance with the invention having a further chamber adjacent to the second chamber, in electrical communication with the second chamber and at a more positive electrical potential than the second chamber. Most simply this may be achieved by providing the anode in a further chamber which is in electrical communication with the second chamber by means of a membrane which is permeable to ions and the chemical compound and hence electric current but impermeable to sperm cells. Negatively charged chemical compound will be drawn out of the second chamber into the further chamber and thereby be separated from the motile sperm cells in the second chamber. A membrane having a molecular cut off value of 100 kDa or less, for example 80, 50, 40, 20 or 10 kDa or less is especially suitable. Such membranes may be manufactured by a variety of techniques using various methods. For example, a polyacrylamide membrane, optionally provided on a support structure may be used.

### Chemical Compounds

The chemical compound(s) for use in conjunction with the apparatus, method or use of the invention will typically be compounds to which the cells have been exposed ex-vivo in order to assist in freezing, or to improve sperm cell quality, for example, motility promoting agents, or to otherwise nourish, protect or otherwise enhance the cell's function, survival or health. It will be advantageous to remove the compounds from the cells before the cells are used for insemination. The cells may have been exposed to more than one compound and a reference to a “compound” as used herein is to be taken to refer to a mixture of compounds. The compound(s) will typically have an electrical charge in solution (under physiologically suitable conditions) which may be positive or negative such that the compound(s) will exhibit electrophoretic migration when exposed to an electrical potential. Compounds may have an inherent electrical charge (for example they may be acids or bases) or a charge may have been introduced by derivatizing the molecule with a charged moiety.

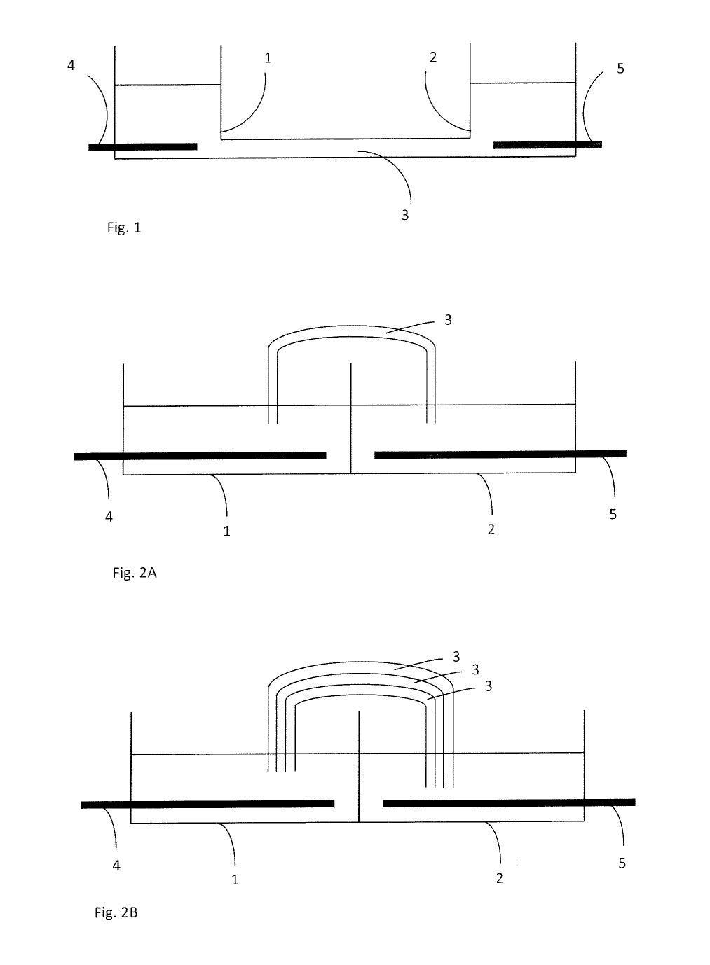

Referring to FIG. 1, the apparatus of the invention may comprise a first chamber (1) and a second chamber (2) which are illustrated diagrammatically only for the purposes of FIG. 1. Channel (3) connects the two chambers and is in communication with both.

The apparatus is so arranged such that channel (3) does not permit significant migration of non-motile sperm cells from chamber (1) to chamber (2). This is accomplished by making channel (3) such that it is sufficiently narrow and long to prevent convection currents from causing significant migration of non-motile sperm cells from the first to the second chamber during a time period which is sufficient for a substantial proportion of motile sperm to swim or be carried by an electrophoretic potential from the first chamber to the second chamber. For example the channel may be dimensioned to permit the passage of no more than 20% of non-motile sperm cells during a time period sufficient to allow the passage of at least 70% of healthy motile sperm cells.

The invention includes an apparatus as defined above or in accordance with the invention which contains in the first chamber a chemical compound as defined herein for sperm cell treatment. The apparatus may optionally be supplied with an appropriate dose of said chemical compound pre-loaded into the first chamber either as a liquid or powder or solid deposited onto one or more of the chamber walls or otherwise dispensed into the first chamber such that when the sperm cells to be separated are placed in the first chamber in an appropriate volume of medium, the chemical compound is present at a concentration suitable to provide its desired action on the sperm cells.

### Number of Channels

According to all apparatuses methods and uses of the invention there are preferably more than one channel linking the first chamber and the second chamber. For example, there may be 2, 3, 4, 5, 6, 7, 8, 9 or 10 (for example 3 to 10 or 4 to 10 or 5 to 8) channels. Multiple channels are advantageous because they allow the invention to work even if one or a few channels become blocked to the passage of sperm cells by cell debris, other debris, a manufacturing defect or an airlock or air bubble.

### Example Chamber Dimensions

Chamber volume (in particular the volume of the first and second chamber) may be, for example, between 0.5 μl and 5 ml, for example between 50 μl and 1 ml, for example between 100 μl and 800 μl, for example from 200 μl to 600 μl, for example approximately 400 μl. The chambers may incorporate means for introducing or withdrawing material from them, for example an open top, lid or port. These volumes relate to both the first chamber and the second chamber and where present any further chambers although in some versions of the invention the preceding volumes relate to the first chamber, the volume of the second chamber being larger so as to accommodate medium for filling the channel(s). Typically it will be larger by approximately the volume of the channel(s), for example 100 to 200 μl larger, 50 to 300 μl larger, 100 to 400 μl larger of 200 to 500 μl larger.

Chambers may be provided ready filled with medium or suitable for filling with medium by the user before use. Preferably at least the second chamber and the channel(s) are filled with medium before use, along with any further chambers which may be present.

According to certain embodiments the first chamber may be provided pre-loaded with a defined quantity of the pre-treatment chemical compound either in solution or alternatively in a solid form, for example as a powder or a coating on the walls of the first chamber.

### Example Channel Dimensions

Considerations influencing the dimensions of the channel(s) include:
channel(s) must be sufficiently narrow and sufficiently long to prevent significant migration of sperm cells along the channel(s) by convection currents during the time the apparatus is in use;
dimensions must support ease of manufacture, for example by injection moulding;
channel(s) must be sufficiently wide and short to allow migration of motile sperm cells along the channel(s) by swimming and/or electrophoresis, during the time that the apparatus is in use.

Typically the apparatus is in use for 1 to 60 minutes, for example from 5 to 60 minutes, for example from 5 to 30 minutes, for example from 5 to 20 minutes. It is also envisaged that the apparatus may be in use for shorter time periods such as 5 to 120 seconds, 10 to 80 seconds or 20 to 60 seconds, or for less than 120, less than 80, less than 60, less than 40 seconds.

The channel dimensions area also be preferably arranged to facilitate efficient and effective filling of the channel(s) with medium. It may be that the distance between opposite channel side walls is such that capillary action causes medium from one or both the first or second chamber to be drawn into the channel(s) by capillary forces acting on the medium so that the channel(s) becomes filled with medium.

The opposite sidewalls of the channels are preferably no more than 1.0 mm, more preferably 0.5 mm apart. The walls are preferably at least 0.1 mm, more preferably at least 0.2 mm apart. The distance between opposite side walls is preferably between 0.1 and 1.0 mm, 0.2 and 0.5 mm or 0.3 and 0.4 mm.

### Use of a Bridge Element

It is a preferred feature of all aspects of the invention that channel(s) are provided as a “bridge element” as described in US 2005/0026274. Briefly an apparatus using a bridge element has the first chamber and the second chamber separated by an intervening wall which is bridged by a bridge element placed over the intervening wall. The bridge element contains the channel(s) of the invention and the open ends of the channel(s) open respectively into the first chamber and into the second chamber thereby providing a path between the two chambers. Preferably the bridge element is arranged so that the ends of the channels placed therein enter the liquid in each chamber to different depths. Differential insertion depths facilitate filling of the channels from the medium or liquid in either chamber but preferably with the medium from the second chamber. Differential insertion depths may be achieved by filling the first and second chambers to different heights and/or by dimensioning the bridge element to extend deeper into one chamber than the other. Preferably the bridge element is inserted more deeply into the second chamber such that when the bridge element is lowered onto the rest of the device, the end placed into the second chamber enters the medium in that chamber first such that liquid in from the second chamber is drawn up into the channel(s) before the other end of the channel(s) enter the liquid in the first chamber so that by the time that they do, any air previously in the channel(s) will have exited the lumen of the channel(s) before both ends of the channel(s) are immersed. Such a consideration may not be necessary if the apparatus is used in methods wherein the liquid is only added to the first chamber after the channel(s) have been filled with liquid.

### Suitable Materials

Preferably the apparatus is transparent. It may be made of glass or plastic but is preferably made of transparent plastic because this allows it to be made cheaply enough to be single use. The plastic should be such that the apparatus may be simply sterilised, for example by electron bombardment. A particularly suitable material may be polycarbonate plastic which has good bio-compatibility and may be injection moulded. An alternative preferred material is polystyrene.

Preferably the apparatus is provided with a lid in order to prevent contamination of the cells.

### Electrode Arrangements

An anode is provided in electrical communication with the second chamber and a cathode is provided in electrical communication with the first chamber. The simplest way to do this is to provide the respective electrodes directly in the respective chambers. Disposable electrodes may be provided as part of the apparatus. For example, carbon electrodes may be provided on the bottom surface of the chambers. Such electrodes may be advantageous in terms of low cost of construction and the fact that they may be incinerated easily as clinical waste after use.

Reusable electrodes may be provided and dimensioned so as to dip into the respective chambers from above. This may optionally be achieved by integrating them into an optional lid or designing them so that a lid reaches over them.

It is important that potentially toxic ions are not released from the electrodes and allowed to come into subsequent contact with the sperm cells. One way of avoiding this would be to construct the electrodes from inert platinum or rubidium of mixtures thereof. The high cost of such precious metal electrodes would mitigate their being single use.

Alternatively, one or both of the electrodes may be provided in separate electrode chambers in electrical communication with the first chamber and second chamber respectively. Such electrode chambers may electrically communicate via ion permeable membranes or salt bridges and be so arranged such that during the time period of the separation process, toxic ions or molecules released from the electrodes or electrolyte do not come into contact with the sperm cells. It will, however, be understood that a certain level of potentially toxic chemical species may be tolerated by the sperm cells or accommodated by the apparatus or method of the invention. For example hydroxide (HU) ions released from the electrolyte may be accommodated by use of a suitable buffer. In embodiments where a separate cathode chamber is provided and the chemical compound is negatively charged, the apparatus may be provided with the chemical compound pre-loaded into the cathode chamber such that under electrophoresis, the chemical compound migrates through the membrane into the first chamber and comes into contact with the sperm cells.

The apparatus of the invention relies on the appreciation that sperm cells and especially healthy, motile sperm cells exhibit sialic acid on their surface and therefore have a negative surface charge. This means that if a voltage is applied to the electrodes, the migration of motile sperm cells along channel(s) (3) will be speeded up which is advantageous because shortened processing times result in lower loss of cell viability. The voltage is applied between the anode (5) and the cathode (4). By integrating the separation of motile sperm cells from less motile sperm cells and other components of semen such as leukocytes with separation from chemical pre-treatment compounds, the number of separate handling steps and the potential for cellular loss therein is reduced.

### Medium

In accordance with the invention, the chambers are filled with ionic buffers (also referred to as “electrolytes” or “media”) in use.

Preferred buffer concentrations are between about 1 to 100 mM. Any suitable buffer or electrolyte can be used. Suitable buffers include, but not limited to, sperm-compatible biological buffers and components such as Ham's, HEPPS, HEPES, BisTris, sodium chloride, phosphate buffer salts, sucrose, glucose and mannitol. As outlined below, a 10 mM buffer of HEPES, 30 mM NaCl and 0.2M sucrose has been found to be particularly useful. It will be appreciated, however, that any other suitable buffer can be used. Suitable buffers for use with mammalian sperm must be “non-capacitating”, in that they do not cause premature capacitation of the sperm cells. Preferably media is present in the second chamber, the channel(s) and any additional chambers if present before the sperm cells are introduced. The first chamber may be empty until the semen sample or other sample containing sperm cells requiring separation (for example a defrosted or processed sample of sperm cells) is added to it. Alternatively, the first chamber may contain medium into which a sample of sperm cells is diluted.

### Electrical Parameters

The strength of the electrical potential and of the current used in the method and uses of the invention and other related parameters may be optimised to the process time, the dimensions of the chambers, the dimensions of the channels and the nature of the sperm cells and chemical compounds and electrolytic buffer. The conditions may in some circumstances be a combination of all or some of the following:
Buffer may be based on a cell culture medium having an osmolarity to 200 to 400 mOsmol/kg, for example 300 to 320 mOsmol/kg, for example 310 mOsm/kg. A suitable buffer may, for example, comprise 10 mM HEPES, 30 mM sodium chloride and 0.2M of sugar such as fructose or sucrose.
pH is preferably 6 to 9, for example 7 to 8, for example pH7.4.
Conductivity may be from 1 to 10 mS/cm, for example 2 to 5, for example 3.8 to 4.2 mS/cm, for example 4 mS/cm.
Current/voltage applied currents of from 20 to 200 ml mA may be suitable. For example from 50 to 150 mA, for example from 60 to 100 mA, for example from 70 to 80 mA, for example 75 mA. A voltage may be applied between the cathode and anode to give an electrical field strength of 10 to 30 V/cm, for example 15 to 20 V/cm, for example 16 to 18 V/cm, for example 17 V/cm. Voltage may be pulsed with time or modulated into any appropriate waveform in which case the values given immediately above are to be understood as mean values averaged out over the entire electrophoretic time period.
Temperature The temperature is preferably chosen for good sperm survival.
Time Preferably the process lasts for a few seconds or minutes for example 10 to 1000 seconds. For example 30 to 300, 50 to 500, 10 to 100 seconds, 20 to 60 seconds, 10 to 120 seconds, or 10 to 40 seconds.

Improving the Zech device by adding electrodes so as to speed up migration of motile cells also allows the apparatus of the invention in be used in a method of treating sperm cells with a chemical compound in order to increase the sperm cells' fertilization capacity, comprising:
A) contacting the sperm cells with a chemical compound in a first chamber of a sperm cell enrichment apparatus wherein said sperm cell enrichment apparatus comprises said first chamber for receiving sperm cells to be selected, separate therefrom a second chamber for receiving the selected sperm cells, and at least one channel extending from the first chamber to the second chamber and having an opening into the first chamber and into the second chamber at its respective ends, the or each channel being dimensioned such that when it is filled with a liquid medium there is negligible movement of sperm cells from the first chamber to the second chamber by convection currents in the liquid medium, but that motile sperm cells are able to swim through the liquid medium from the first chamber to the second chamber, characterised in there is negligible movement of the chemical compound from the first chamber to the second chamber either by convection or diffusion, and
B) applying a voltage between the first chamber and the second chamber such that the first chamber is placed at a more negative electrical potential than the second chamber;
C) waiting until at least a portion of motile sperm cells have migrated from the first chamber to the second chamber by swimming and/or electrophoresis wherein said chemical compound is retained in the first chamber or captured in a further chamber separate from the sperm cells in the second chamber.

Optional features of methods of the invention may be as described herein by reference to the apparatus of the invention and/or as described below with reference to FIGS. 1 to 3.

### Sperm Cells

The sperm cells for use in the invention will typically have been collected from a male animal (for example a human subject) by any suitable method including masturbation, prostate massage, the use of an artificial vagina (for example as part of a breeding mount used for collection from male horses, cattle or other non-human animals), vibroejaculation and electroejaculation. Under some circumstances collection may involve use of a collection condom or retrieval directly from testes by testicular sperm extraction (TESE) may be used. Collection involving ejaculation is generally favoured because it will result in a sample of sperm cells suspended in semen and therefore more likely to be properly matured. The sperm cells may be collected “fresh” or may have been collected previously and frozen for a period of storage and then thawed when required for use. They may optionally be subjected to a pre-treatment step before use in a process or method of the invention. Optionally the cells may have been subjected to a preceding treatment, washing or dilution step. It may, however, be possible to dispense with a pre-washing step because the separation step of the invention results in cells in the second chamber which have effectively been “washed” of components remaining in the first chamber. Such an arrangement may be advantageous because it allows elimination of a separate washing step which allows a reduction in the number of processing steps and an overall reduction in total sperm cell processing time. In such methods, freshly obtained semen or a newly-thawed sample of sperm cells may be placed directly into the first chamber.

A further embodiment of the invention there is provided use of sperm cells separated from a chemical compound according to a method of the invention for use in intrauterine insemination (IUI), intracervical insemination (ICI) in-vitro fertilisation (IVF) or intra cytoplasmic sperm injection (ICSI).

### Subsequent Use of Sperm Cells

The process of the invention optionally includes the post-separation step of removing the sperm cells from the second chamber, optionally assessing sperm quality (for example using a viability assay such as an eosin exclusion assay, a DNA damage assay such as a TUNEL assay, a mobility assay or simply a cell count), optionally formulating the cells in a suitable medium and then “loading” the cells into a device suitable for insemination, for example intrauterine insemination. Optionally the process of the invention further includes the step of carrying out insemination of a female animal or woman, for example by intrauterine insemination. Sperm cells processed in accordance with the first aspect of the invention may be loading into a device suitable for use in ICI, IUI, IVF or ICSI. Optionally, the process of the invention further includes the step of using the sperm cells in an ICI, IUI, IVF or ICSI procedure.

Methods of the invention involve the use of an apparatus comprising at least two chambers, a first chamber (1) and a second chamber (2). The first and second chambers are linked to each other by at least one channel (3) which opens at its respective ends into the first chamber (1) and the second chamber (2). The first chamber is in electrical communication with a cathode (4) and the second chamber is in electrical communication with an anode (5). The cathode and anode are shown in FIG. 1 as being directly provided in, respectively, the first chamber and the second chamber and this is the simplest configuration.

When in use in a method of the invention, the channel(s) and the second chamber contain medium. Medium may also be provided in the first chamber so that there is fluid communication from the first chamber to the channel (3). This fluid communication may alternatively be provided by a fluid containing sperm cells (for example seminal fluid) in the first chamber but it may also be provided in part by the addition of medium, particularly if the volume of the fluid containing sperm cells is not on its own sufficient for fluid communication to be established between the first chamber and the second chamber via the channel(s).

The chambers and the channel(s) may be dimensioned and constructed as described above in reference to the apparatus of the invention.

Motile sperm cells are able to swim along the channel(s) and thereby migrate from the first chamber to the second chamber through the medium therein. Less motile sperm cells remain in the first chamber (as do other cell types such as leukocytes and cellular debris). This means that after an incubation step of typically about 10 to 40 minutes, the sperm cells in the second chamber are enriched for motile sperm cells. Methods of the invention may optionally include the step of removing sperm cells from the second chamber and using them in a subsequent process such as fertilization or insemination.

However, when a voltage is applied between the cathode and the anode in accordance with the invention, the migration of motile sperm cells into the second chamber is sped up. This is because healthy motile sperm exhibit higher levels of sialic acid on their cell surfaces than other cells and are therefore carried to the second chamber by electrophoresis in addition to their swimming. This “electrophoretic assistance” allows the process time to be cut to typically only a few seconds or minutes (for example 10 to 120 seconds, 20 to 60 seconds or 10 to 40 seconds). Shorter processing times mean less loss of cell viability. The cell migration step of the invention is typically carried out at a temperature supportive of sperm cell survival. It may be necessary to take into account increases in the temperatures of the fluids due to the passage of the electric current. In some embodiments the apparatus is thermostatically controlled to prevent it becoming too hot. In other embodiments the apparatus may be cooled by placing it into a relatively large volume of water at a desired temperature. However, because methods of the invention are generally performed in only a few seconds it is not usually the case that excessive temperatures will become problematic. Sperm cells are reasonably tolerant of temperatures between 20 and 40° C., preferably 23 to 37° C. and preferably the invention will be performed within that temperature range. Although temperatures at the lower end of this range are perfectly acceptable for sperm survival, higher temperatures may help sperm cells swim faster. Accordingly, in some embodiments the temperature will be between 25 and 40° C., more preferably between 26 and 39° C., 27 to 38° C., 30 to 38° C., 32 to 38° C., 35 to 37° C. However, lower starting temperatures may be preferable if it is anticipated that the flow of the electrical current will significantly elevate the temperature. Accordingly in some embodiments the temperature, for example the starting temperature, will be between 20 and 35° C., 22 and 33° C., 22 and 32° C., 22 and 30° C., 22 to 28° C., 23 to 27° C. or 22 to 25° C.

Methods of the invention also include the initial step of contacting the sperm cells with a chemical compound before the step of electrophoretic cell enrichment. The sperm cells introduced into the first chamber will be in contact with the chemical compound. The chemical compound may have been brought into contact with the sperm cells in that chamber or it may have been brought into contact with the sperm cells prior to their introduction into the first chamber.

If the chemical compound has no overall electrical charge it will exhibit no electrophoretic mobility and may therefore be separated from the motile sperm cells which migrate to the second chamber by virtue of remaining in the first chamber when the motile sperm cells migrate along the channel(s). For this variation of the invention to work, it is necessary that the sperm cell separation step is sufficiently quick and that the chemical compound is sufficiently slow to defuse such that a material amount of the chemical compound does not diffuse along the channel(s) during the method of the invention. It is also advantageous in this regard that the channel(s) are sufficiently long (for example at least 5 mm or at least 10 mm or at least 20 mm long) and sufficiently narrow (for example between 0.1 and 0.5 mm, 0.2 and 0.4 mm or 0.3 and 0.4 mm narrow) for diffusion during the time period of the method to be negligible and for convective mixing of the chemical compound with the medium in the channel(s) to not take place to a significant degree.

If the chemical compound has a positive charge in solution (either naturally or because of a deliberate derivatization with positively charged moieties) the use of an electrophoretic current brings additional advantages in that the positively charged compound will be electrophoretically prevented from migrating through the channel(s). In such methods it is preferred that the current remain switched on until the separated motile sperm cells are withdrawn from the second chamber of the apparatus.

FIG. 2, shows diagrammatically the use of channels incorporated in a bridge element in accordance with apparatuses, methods and uses of the present invention. FIG. 2A shows the side-on view of an apparatus of the invention comprising the first chamber (1) and the second chamber (2), cathode (4) and anode (5) as before. However the channel is provided as a bridge element which extends from the first chamber to the second chamber. According to some embodiments, the bridge element may be placed in fluid communication with the first chamber and the second chamber after those chambers have been filled with medium and/or sperm cells. Methods of the invention optionally include the step of placing the bridge element. Optionally the bridge element may be provided integrated into a lid which is placed over the chambers after filling (lid not shown in FIG. 2A).

FIG. 2B shows diagrammatically how multiple channels may be provided to allow multiple fluid paths between the first chamber and the second chamber. It also shows that the channels are arranged to extend deeper into the second chamber than they are into the second chamber. This provides the advantageous feature that when an air-filled bridge unit is placed into the fluid filled chambers, capillary forces cause the channels to fill with medium from the second chamber. This feature may of course be also used in single channel devices and methods. Although not shown in FIG. 2B, multiple channels are typically of similar or identical dimensions to each other. Specifically multiple channels may be of substantially the same length as each other.

Although for simplicity the figures of the present patent application show the first chamber and second chamber as being provided side-by-side, they may be provides in other arrangements. For example, one chamber may be concentric to the other, for example as illustrated in US2005/0026274.

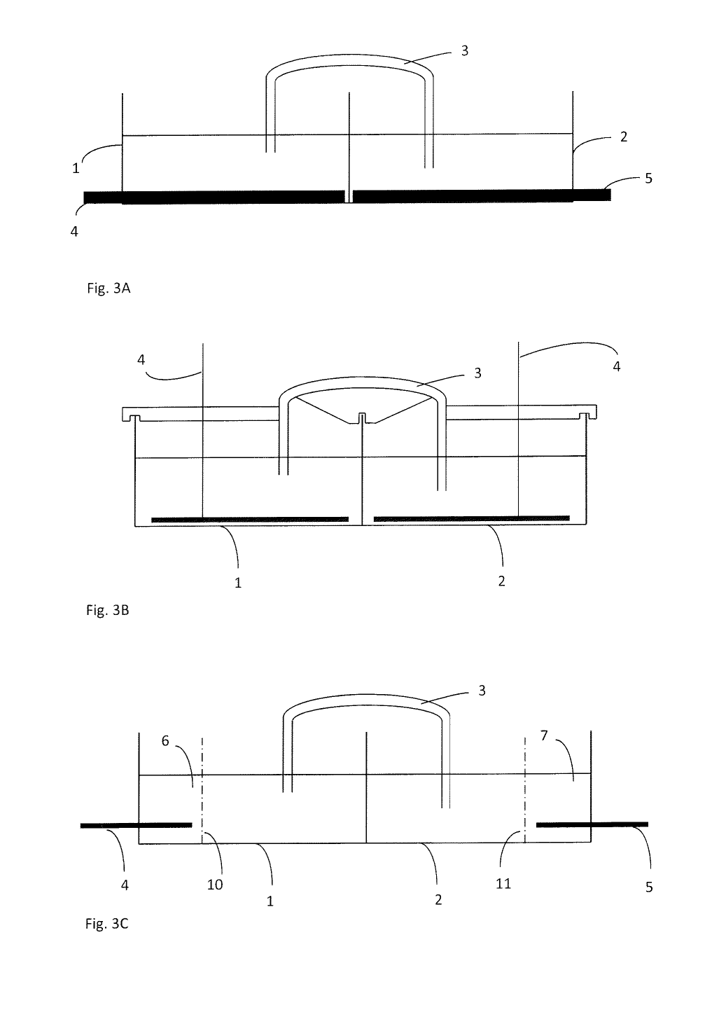

FIGS. 3A to 3C show various alternative arrangements of electrodes. It is to be understood that these electrodes arrangements may be combined with the various channel arrangements disclosed herein. FIG. 3A shows carbon electrodes integrated into the bottom of the chambers. Such an arrangement may be acceptably inexpensive for the apparatus to be fully disposable and may permit apparatus which is fully incineratable after use. Alternatively, electrodes may be reused between patients and sterilised between uses. Platinum/Rubidium electrodes would probably be treated as reusable items because of their cost. Electrodes may therefore be dipped into the liquids in the first and second chambers from above as shown diagrammatically in FIG. 3B. FIG. 3B also shows the optional feature of integrating the electrodes with an optional lid (6).

It will be preferable to choose electrodes and electrolytes which are sufficiently harmless to sperm cells so as to not negatively impact on cell health during the time period that they are in the apparatus. If that is not done it may be necessary to provide one or more further chambers for housing the electrodes. FIG. 3C shows an arrangement whereby the cathode is housed in a further chamber (6) and the anode is housed in separate further chamber (7). The invention also encompasses apparatuses, methods and uses having only one of the further chambers shown in FIG. 3C. In accordance with the invention, electrical communication between the first and second chambers and their respective further chambers is provided by ion permeable membranes. Alternatively it may be provided by means of a salt bridge between the respective chambers

The arrangement shown in FIG. 3C may be used with separation methods involving positively charged chemical compounds. The positively charged chemical compounds may be retained in the first chamber or migrate electrophoretically into the further chamber housing the cathode. The arrangement shown in FIG. 3C may also be used when the chemical compound is charge-neutral.

The arrangement shown in FIG. 3C is however especially suitable for use with negatively charged chemical compounds when the ion permeable membrane between the second chamber and the further chamber housing the anode is impermeable to sperm cells but permeable to the chemical compound. This allows the chemical compound to be segregated into the further chamber and thereby be separated from the motile sperm in the second chamber.

1. An apparatus for separating sperm cells from positively charged motility promoting agents, comprising a first chamber for receiving the sperm cells to be selected, separate therefrom, a second chamber for receiving the selected sperm cells, and at least one channel extending from the first chamber to the second chamber and having an opening into the first chamber and into the second chamber at its respective ends, the channel being dimensioned such that when it is filled with a liquid medium there is negligible movement of sperm cells from the first chamber to the second chamber by convection currents in the liquid medium, but that motile sperm cells are able to swim through the liquid medium from the first chamber to the second chamber; wherein the apparatus is provided with a cathode in electrical communication with the first chamber and an anode in electrical communication with the second chamber such that a voltage may be provided between the first chamber and the second chamber thereby assisting the migration of sperm cells from the first chamber to the second chamber by electrophoresis.

2. The apparatus as claimed in claim 1, wherein the cathode is provided in a further chamber in electrical communication with the first chamber via an ion permeable membrane and wherein the cathode is provided in another further chamber in electrical communication with the second chamber.

3. The apparatus as claimed in claim 1, wherein the cathode is provided in a further chamber in electrical communication with the first chamber via an ion permeable membrane and wherein the cathode is provided in another further chamber in electrical communication with the second chamber.

4. The apparatus as claimed in claim 1, wherein the anode and cathode are platinum/rubidium electrodes

5. The apparatus as claimed in claim 1, comprising at least 2 channels

6. The apparatus as claimed in claim 1, wherein said channel(s) are provided as a bridge element which is placed over the first chamber and second chamber and which extends into the respective chambers.

7. The apparatus as claimed in claim 6, wherein the bridge element extends into the second chamber to a greater depth than into the first chamber.

8. The apparatus as claimed in claim 1, wherein the chambers and channel are constructed of transparent polycarbonate plastic material.

9. The apparatus as claimed in claim 1, wherein the first chamber contains a positively charged motility promoting agent for increasing sperm cell fertilization capacity.

10. A method of separating motile sperm cells from non-motile sperm cells, the method comprising using the apparatus of claim 1, wherein motile sperm cells are simultaneously separated from a positively charged motility promoting agent previously placed in contact with the sperm cells.

11. (canceled)

12. A method of treating sperm cells with a positively charged motility promoting agent in order to increase the sperm cells' fertilization capacity, comprising:
A) contacting the sperm cells with a positively charged motility promoting agent suitable for increasing the sperm cells fertilization capacity in a first chamber of a sperm cell enrichment apparatus wherein said sperm cell enrichment apparatus comprises said first chamber for receiving sperm cells to be selected, separate therefrom a second chamber for receiving the selected sperm cells, and at least one channel extending from the first chamber to the second chamber and having an opening into the first chamber and into the second chamber at its respective ends, the or each channel being dimensioned such that when it is filled with a liquid medium there is negligible movement of sperm cells from the first chamber to the second chamber by convection currents in the liquid medium, but that motile sperm cells are able to swim through the liquid medium from the first chamber to the second chamber; and
B) applying a voltage between the first chamber and the second chamber such that the first chamber is placed at a more negative electrical potential than the second chamber; and
C) waiting until at least a portion of motile sperm cells have migrated from the first chamber to the second chamber by swimming and/or electrophoresis wherein said positively charged motility promoting agent is retained in the first chamber.

13. (canceled)

14. (canceled)
